(19) United States
(12) Patent Application Publication
(10) Pub. No.: US 2009/0319023 A1
(43) Pub. Date: Dec. 24, 2009

(54) Stents and Stent Grafts

(76) Inventors: Daniel K. Hildebrand, San Francisco, CA (US); Nicholas C. deBeer, Montara, CA (US); Maria G. Aboytes, Palo Alto, CA (US); Arturo S. Rosqueta, San Jose, CA (US); Chi Vu, Palo Alto, CA (US); Siddharth Loganathan, Mountain View, CA (US); Frank P. Becking, Santa Clara, CA (US)

Correspondence Address: ORRICK, HERRINGTON & SUTCLIFFE, LLP;IP PROSECUTION DEPARTMENT, 4 PARK PLAZA, SUITE 1600, IRVINE, CA, 92614-2558, US

(21) Appl. No.: 12/397,123

(22) Filed: Mar. 3, 2009

Related U.S. Application Data
(60) Provisional application No. 61/035,328, filed on Mar. 10, 2008.

(51) Int. Cl. A61F 2/06

(52) U.S. Cl. 623/1.13; 623/1.16

(57) ABSTRACT

The subjected devices include a stent, a graft and a means for attaching the graft to the stent. One or more members are received in a permanent or temporary receptacle within the stent attach the graft to the stent. In one variation, an interference fit is employed; in another, the graft is bonded to a stent-captured member(s).

### CROSS-REFERENCE TO RELATED APPLICATIONS

The present application claims priority to U.S. Provisional Patent Application Ser. No. 61/035,328, filed Mar. 10, 2008, entitled “Stent-Grafts,” which is fully incorporated by reference herein.

### BACKGROUND

Stent grafts have use in a variety of applications. However, placing a graft on a stent can reduce the overall longitudinal flexibility of the implant as compared to a bare metal stent, and successfully affixing or attaching the graft to the stent has been problematic. Rings or other retaining members have been used on the outside of grafts to hold them to stents. However, these approaches result in devices having a relatively large outside diameter (OD). The rings can also cause delivery problems due to vessel lumen contact with the retaining members.

Graft retention has also been attempted without the use of retainers. For example, U.S. Pat. No. 6,214,039 to Banas, et al. discloses a balloon-expandable stent graft employing ePTFE as a cover. The graft is circumferentially engaged about a stent and is retained thereon by a radial recoil force exerted by the tubular graft against the stent. The graft is thereby retained on the stent (or stents) without the use of adhesives, sutures or other attachment means. The covered stent is assembled by joining a dilation mandrel and a stent mandrel, placing the graft on the dilation mandrel where it is radially expanded and passing the expanded graft over the stent that is positioned on the stent mandrel. However, system safety is questionable since the graft material is not secured to the stent in any other way. Indeed, because preload applied to an ePTFE graft layer may tend to decay to zero (e.g., while the device is stored), instances may occur in which no preload is left on the material to keep the graft secured when navigating tortuous anatomy.

U.S. Pat. No. 6,086,610 to Duerig, et al. discloses a related approach for a self-expanding stent with the addition of a storage sheath. While graft relaxation under the constant pressure of the stent might be avoided by such an approach, it still raises questions of whether the stent will cut into the graft as the ePTFE creeps due to constrained strut contact. Such creep could result in holes or tears in what should be an imperforate body.

In effort to provide a low-profile stent graft solution, the reference systems avoid the use of retainers that extend beyond the outer boundary of the graft. They also avoid bulky multilayer graft “sandwich” type attachment techniques (e.g., as disclosed U.S. Pat. Nos. 5,700,285, 5,735,892 and 5,810,870 to Myers, et al.) However, a need persists for constructions ensuring long-term reliability without compromise to flexibility and/or compressibility. The present invention meets these needs and others, including providing an improved stent scaffold pattern, especially for use in stent graft constructs.

### SUMMARY OF THE INVENTION

The present invention includes stents and stent grafts with the grafts variously retained upon the stents. A stent portion of the stent graft construct is broadly characterized as a tubular lattice support structure or scaffold having a plurality of cells. Depending on the mode of action, the stent material may be ductile/distensible (thus, balloon-expandable) or elastic or super-elastic/shape memory alloy (thus, self-expanding). The graft portion of the subject devices may partially or fully cover the stent (from a radial and/or axial perspective). While any suitable combination of stent and graft configuration may be used with the graft-to-stent retention features of the present invention, the invention also provides particular stent graft constructs having functional advantages beyond their inter-retention capabilities, as discussed in greater detail below.

The subject implants are designed to reliably secure graft material to a stent structure in a way that minimizes or altogether avoids additional thickness to the final product. The graft-to-stent attachment may be accomplished through either an interference fit mechanism using plastically-deformable or malleable members (e.g., metal pins) pressed into receptacles in the stent design to capture the graft, or with polymer (e.g., fluorinated ethylene polypropylene (FEP)) members or blocks that are heat or chemically bonded (e.g., by solvent or adhesive) to the graft and either permanently or releasably received within receptacles in the stent. Generally, the members retained by the stent and holding the graft may be regarded as attachment bodies. When they are pressed-in, trapping the graft within the receptacles, they may be regarded as interference bodies. When directly bonded to the graft, or bonded thereto using an intermediate adhesive or medium, they may be regarded as bonding bodies.

Depending on the stent graft attachment method, the receptacles may be openings or eyelets formed within the stent discrete from the normal/repeating stent cell structure. Otherwise, they may be designated cells within the stent's lattice structure. The receptacles may be used to permanently retain an attachment body or may be used only temporarily to retain apposition of the graft to the stent scaffold prior to and during deployment at a target site. Any combination of these arrangements may also be employed.

In the variations of the invention in which the receptacles are permanently set within the stent pattern in the form of eyelets, they may be incorporated into the pattern in a manner similar to the dedicated marker receptacles as described in U.S. Pat. No. 6,022,374 to Imran. Namely, the structures may be formed separate and distinct from the functional geometry of the stent cells.

Generally, an eyelet comprises a rim and receptacle region. For example, the rim may be an ovoid or circular shape with an open space in the therein. It may include additional grip features within its field. Preferably, though not necessarily, the eyelet region(s) is/are formed in the stent pattern at the same time the stent is laser cut from tubular stock.

The eyelet or receptacle regions may be configured to receive interference bodies that may be made of tantalum, gold, platinum, alloys thereof or other material. Graft attachment is achieved by trapping or setting the graft material between an interference body and the eyelet rim through an interference fit. When such an interference fit is desired, the eyelets are advantageously round in shape and the interference bodies are in the form of cylindrical pins or pucks. However, other shapes—such as spheres that are subsequently flattened—may be employed. Indeed, spherical bodies may offer certain advantages by self-centering in the receptacle regions. Otherwise, appropriate fixturing may be employed as an aide.

The interference bodies are preferably radiopaque and ductile. Radiopacity allows for radiologic visualization of the implant during and after device deployment by use of the attachment bodies alone. Especially when serving dual-use as marker plus attachement features, the bodies will typically be set at or adjacent (at least) the ends of the graft and/or stent. However, they may be used at any suitable location on the device. Ductility of the interference members allows them to conform around any receptacle features provided to enhance interference and/or slightly “mushroom” or “head” along an inner periphery of the receptacle. The strength offer by metal bodies so-processed may be desirable. However, polymeric bodies may be similarly employed in forming an interference fit to retain the graft.

In other embodiments, the eyelet/receptacle may be used to retain an attachment body that is attached to the graft material along only the surface of the body. Especially when the bonding body is a polymer (e.g., FEP) puck, plug, or block, it can be heated to directly bond to the graft.

While the graft material changes configuration by opening or stretching upon stent expansion, the bonding bodies within the eyelet(s) may remain substantially stationary. In certain variations (e.g., where the attachment bodies are bonded within a surrounding eyelet or receptacle), they typically remain retained within the eyelets post-deployment. With this approach to inter-retention of the graft and stent, a greater variety of eyelet shapes is available, including both regular (e.g., circular, square, hexagonal, etc.) and irregular (e.g., semi-circular, rectangular, etc.) shapes.

Post-deployment retention of the polymer blocks within the stent may not however, be necessary as the apposition of the stent graft in the vessel upon implantation is often sufficient to retain the positions of both the stent and graft. As such, another variation of the invention employs only temporary retention of the attachment bodies. As such, the stent cells themselves may suffice as temporary receptacles for the polymer members, thereby eliminating the need to form designated eyelets within the stent lattice. When the stent is compressed, the cells can form interstices or pockets to retain the bonding bodies until the stent geometry changes shape upon stent expansion.

The attachment bodies holding the graft are retained within these stent regions to secure the lateral/axial location of the graft relative to the stent until released. The graft-retaining bodies are released from direct contact with the struts or cells of the stent upon stent expansion (by balloon inflation or restraint release), but they continue to be retained by the overall implant by virtue of their permanent bond to the graft material. The graft material may be retained in contact with the stent as it is stretched during stent expansion. Finally, contact with the vessel wall ultimately secures graft/stent position when the delivery system is withdrawn.

When using polymer-member graft attachment bodies, one or more radiopaque markers may be employed in separate receptacles for identifying the device under medical imaging. The radiopaque markers can also hold the graft to the stent (as detailed above), or alternatively, can operate merely to identify the position of the stent graft radiologically. Yet another option is to load the polymer retainers with radiopaque material such as iodine or tantalum powder.

With the various approaches to graft retention described herein, at least one distal graft connection point is employed. More typically, a plurality of connection points, regions or sections are utilized, often around a circumference of the stent. Both proximal and distal connection points are advantageously employed so that neither end of the graft is prone to migration during advancement or retraction in achieving ideal placement. Moreover, medial connection points may also be employed. Such connection points may offer further stability/support to the graft. It is also contemplated that the graft may be secured to either the exterior or the interior of the stent, with attachment bodies applied accordingly.

With balloon expandable stent based variations of the invention, the graft covering may expand plastically with the stent upon balloon inflation, but does not need to be oversized relative to the stent or to the balloon. In self-expanding variations (i.e., with elastic, super/pseudoelastic or SMA metal stents), the graft material will typically be sized at or closer to the expanded diameter of the device. Thus, for delivery, the additional graft material present when the stent is in an unexpanded state may be folded in a manner as often used to compress and fold balloons for percutaneous angioplasty (see, e.g., U.S. Pat. No. 5,792,172 to Fischell, et al. and U.S. Pat. No. 6,013,092 to Dehdashtian, et al.—both incorporated by reference in their entireties). Upon expansion of the angioplasty balloon, the folds open. Similarly, with the subject self-expanding stents, the material of the graft can be folded against the collapsed stent. Then, when the self-expanding stent opens, the folds open to accommodate the expanding stent.

For balloon-expandable stents in which ePTFE graft material is used in such a way that it plastically deforms from a minimum diameter to a final size, the graft material is typically between about 0.002 and about 0.005 inches thick. However, other materials are contemplated for both balloon-expandable and self-expanding versions of the device. Namely, the graft material can be made of a material selected from silicones, e.g., silicone rubbers, synthetic rubbers, polyethers, polyesters, polyolefins, modified polyolefins, polyamides, fluorinated ethylene propylene copolymer (FEP), polyfluorinated alkanoate (PFA), polyurethanes, segmented-polyurethanes, segmented polyether-polyurethanes, polyurethaneurea, silicone-polyurethane copolymers, and, any analogs, homologues, congeners, derivatives, salts and combinations thereof. Preferred graft material is expanded poly-tetra fluoro ethylene (ePTFE), NiCast™, spun urethane, fine braids (e.g., braids of polymer, metal, plastic, or NiTi). Still other materials or composites including the above-referenced materials may be used in the present invention. Naturally, the optimal thickness of the graft material will also depend on the intended use.

Receptacle and/or attachment body size may typically be between about 0.010 to about 0.025 inches in diameter. Yet, they may be smaller or larger—the latter, especially when for use in non-neuro applications. In any given stent graft, the receptacles and/or attachement body size can be the same throughout, or varied (e.g., especially in those stent grafts utilizing both types of attachement bodies).

The polymer blocks, pucks or plugs forming the bonding bodies placed in receptacles can be a material other than FEP. However, FEP offers an advantage of being heat bondable/attachable directly to ePTFE. Still, an intermediate bonding material (e.g., biocompatible glue such as N-butyl cyanoacrylate (NBCA)) can be used to connect suitable substrates. Likewise, a polymer such as FEP could be delivered in liquid form like “hot melt” glue into permanent or temporary receptacles to secure the stent and graft. In any case, all materials involved will typically be biocompatible, resorbable, and/or biodegradable to the human body.

In addition, while the interference/press-fit approach described usually makes reference to using metal bodies, high-strength polymer members can be used instead. A polymer such as PEEK can offer sufficient structural interface to retain position within the receptacle and hold the graft.

The stent lattice support structure can be formed from a variety of different material in either balloon expandable stents or self-expanding form. An survey of potentially applicable stent constructions can be found in an article published by Nitinol Devices and Components (NDC) located in Fremont, Calif., titled, “A Survey of Stent Designs” by D. Stoeckel et al. Min Invas Ther & Allied Technol 2002: 11(4) 137-147, which is hereby incorporated by reference in its entirety. Bi-stable stent technology as described in U.S. Pat. No. 6,488,702 to Besselink, also incorporated by reference in its entirety, may also be employed. A stent comprising SMA can be self-expanding or balloon expandable. Examples of the former are well known. Examples of the latter are provided in U.S. Pat. No. 5,733,330 to Williams and U.S. Pat. No. 5,766,239 to Cox, each incorporated by reference in its entirety.

While any suitable stent pattern may be used with the graft retention features of the present invention, the invention also provides a unique stent lattice structure which is highly flexible when in a closed or compressed condition, yet provides superior support to the graft material when in an open or expanded condition. In a closed condition, the stent struts are highly curved, providing enhanced flexibility particularly along the longitudinal axis of the stent When open, the stent struts arrange themselves to provide repeating cells having a roughly rhomboid shape. While the segments of the open rhombus structure are substantially identical in shape, they are not when the stent is closed or compressed. Rather, they are optimized for delivery trackability.

Various therapeutic agents may be used in or on the stent graft, particularly the graft portion of the implant—including but not limited to antibiotics, anticoagulants, antifungal agents, anti-inflammatory agents, antineoplastic agents, antithrombotic agents, endothelialization promoting agents, free radical scavengers, immunosuppressive agents, antiproliferative agents, thrombolytic agents, and any combination thereof. The therapeutic agent may be coated onto the stent graft implant, or onto the graft only, mixed with a biodegradable polymer or other suitable temporary carrier and then coated onto the stent graft implant, or the graft alone, or, when part of the implant is made from a polymeric material, dispersed throughout the polymer. The agent can be directly applied to the graft or stent surface(s) as a continuous coating or in discrete droplets, introduced into pockets or an appropriate matrix set over at least an outer portion of the stent. For example, in the case where an aliquot of hydrogel is placed within the space occupied by crossing members, the hydrogel can be impregnated with one or more therapeutic agents that deliver drug to the aneurysm and surrounding vascular tissue. The therapeutic agent may also be covalently attached to the graft material or the stent graft.

The invention further comprises several methods. One set of methods contemplates modes of retaining bonding bodies in the stent. Such retention is accomplished using the graft material and its attachment to the polymer bodies through the stent. In one method, to accomplish this retention, the tubular lattice support structure (e.g., the stent) is compressed. Compression of the stent produces receptacles (at least) at the stent ends (typically distally and proximally, but also medially if desired) and each of these receptacles can receive a polymer body within it. The polymer, such as FEP, may be placed in the receptacle region in a molten or semi-molten state and allowed to cure to fill the receptacle. Precut polymer (e.g., FEP) blocks can also be placed in the receptacles. Once the compressed stent is thus prepared with available receptacles filled, each of the polymer bodies is heat bonded to the graft that overlays the stent. The resulting stent graft expands during deployment which action retains the polymer bodies within the stent and so also within the entire stent graft.

When using either type of receptacle (i.e., separately formed eyelets or compressed lattice cells), heat bonding can be used to locally heat the bonding body and melt it into the graft material. The process of heat bonding can be accomplished either from outside the stent, or from within the lumen of the stent, or both. An exemplary heat bonding device is a temperature-controlled soldering iron with a flattened tip.

In a method of making a stent graft with interference-type attachment bodies, it is important that the retain a shape into which the interference body can be pressed. Ideally, the body material plastically deforms with the receptacle to lock-in with any features provided therein and/or with the graft. Typically, at least some of the pins in stent grafts using this graft securing method are radiopaque markers such as a gold or platinum.

As with the other variations of the stent graft, in the practice of the method, the support structure or stent can be balloon expandable, or self-expandable. For balloon expanding embodiments, in one method of treatment, upon reaching an aneurysm, the balloon is expanded to cause the stent to expand, often to its fullest capacity, and to stretch the graft tightly around the stent. The self-expanding stent grafts are delivered as is customary for self-expanding stents otherwise, (i.e., within a catheter or delivery sheath). The graft material is folded around the crimped stent, and stent and graft are placed within the catheter. Once the stent is placed at the aneurysm (and released from the catheter) the stent expands to stretch the graft material and fit snuggly at the site of the aneurysm. Radiopaque features allow the practitioner to guide both types of stent into place.

The present invention specifically includes combinations of features of various embodiments as well as alternative combinations of the various embodiments where possible, in addition to those features, embodiments, and combinations already specifically described.

### BRIEF DESCRIPTION OF THE DRAWINGS

The figures provided herein are not necessarily drawn to scale, with some components and features being exaggerated for clarity. Each of the figures diagrammatically illustrates aspects of the invention. Of these:

FIG. 1 illustrates an exemplary stent cut pattern for a balloon-expandable stent;

FIG. 2 illustrates an exemplary self-expanding stent;

FIG. 3 is a perspective end view of one variation of a stent graft including bonding bodies received in receptacles provided in the ends of a stent like that in FIG. 1 or FIG. 2;

FIGS. 4A-4C depict additional variations of stent grafts according to the present invention utilizing stent-based retention receptacle features;

FIGS. 5A and 5B illustrate alternative balloon-expandable stent cut patterns with independent receptacle regions, and FIG. 5C is an enlarged view of a single eyelet receptacle from either figure;

FIGS. 6A and 6B illustrate stent grafts of the present invention in which bonding bodies and interference bodies, respectively, are retained within eyelet receptacles of the stent pattern of FIG. 5A or 5B;

FIG. 7 is partial cut-away view of a stent graft of the present invention in an expanded condition upon a balloon;

FIGS. 8A-8C illustrate a portion of a stent body according to the present invention in an as-cut diameter, compressed (as on a balloon catheter) and in an expanded diameter, respectively;

FIG. 9 shows a stent graft having the stent pattern of FIGS. 8A-8C in operative use to treat an aneurysm in a model;

FIGS. 10A and 10B illustrate a novel graft arrangement of the present invention to accommodate high strains around bends;

FIGS. 11A and 11B illustrate a bare stent and a stent graft, respectively, deployed within a vessel adjacent aneurysms;

FIGS. 12A and 12B depict delivery systems for self-expanding and balloon-expandable stents and stent grafts, respectively;

FIGS. 13A-13E depict acts performed during delivery and deployment of a stent graft when using the delivery system embodiments of FIGS. 12A and 12B, respectively;

FIG. 14 depicts exemplary distal, proximal and medial attachment of the graft to the underlying stent; and

FIG. 15 is a flowchart of relevant portions of an exemplary method for fabricating the stent grafts of the present invention in which the grafts are affixed to the stents by way of polymer bonding bodies.

Variations of the invention from those embodiments pictured are contemplated. Accordingly, depiction of aspects and elements of the invention in the figures is not intended to limit the scope of the invention.

### DETAILED DESCRIPTION

Various exemplary embodiments of the invention are described below. Reference is made to these examples in a non-limiting sense. They are provided to illustrate more broadly applicable aspects of the present invention. Various changes may be made to the invention described and equivalents may be substituted without departing from the true spirit and scope of the invention. In addition, many modifications may be made to adapt a particular situation, material, composition of matter, process, process act(s) or step(s) to the objective(s), spirit or scope of the present invention. All such modifications are intended to be within the scope of the claims made herein. The present application claims priority to U.S. Provisional Patent Application Ser. No. 61/035,328, filed Mar. 10, 2008, entitled “Stent-Grafts,” which is fully incorporated by reference herein.

The graft in the stent graft device can be attached to the stent in a number of different ways. The figures serve to illustrate some of these configurations of the stent graft and some of the details of the embodiments. Generally, some features are represented in one figure that may apply to another figure in a differently configured embodiment. Broadly there are two basic attachment body approaches: one that involves bodies that are press-fit into a receptacle or eyelet in the stent to grasp the graft to the stent, and one that involves using bodies that are bonded to the graft material to secure the graft to the stent. Both of these approaches can be used to secure grafts to either self-expandable or balloon-expandable stents.

In either case, the stent lattice support structure can be formed from a variety of different geometries and patterns including cells formed by struts, coils, weaves, and other lattice arrangements. Various stent types and stent constructions that may be employed in the invention regardless of the patterning on the stent. Some more specific options and variations of the invention are embodied and depicted in several Figures.

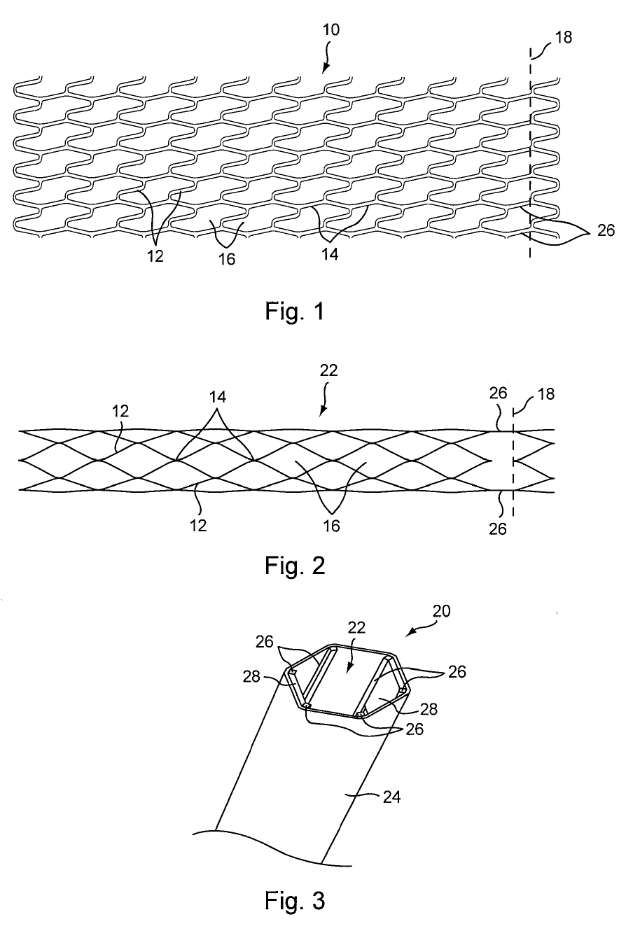

Of these, FIG. 1 illustrates an exemplary stent cell pattern 10 for a balloon-expandable stent suitable for use in the present invention. The stent pattern is shown flattened/unrolled and formed by a plurality of serpentine shaped struts 12 (forming rings when the stent is in its tubular form) interconnected to each other by bridging elements 14 which extend between the curved portions of the struts. The collective structure provides a plurality of closed cells 16. FIG. 2 shows an intact stent 22. It also incorporates struts 12 and may incorporate discrete bridge segments 14 between adjacent cells 16 or not. Each stent design may be employed in the subject stent grafts in either a balloon expandable or self-expanding product. Material selection (as noted above) and further design nuance will apply as appreciated by those with skill in the art.

Moreover, each of the these stent designs is suitable for variations of the stent grafts of the present invention, such as stent graft 20 illustrated in FIG. 3. Stent graft 20 utilizes polymer bonding body type retention features to maintain retention of the stent 22 and graft 24 relative to each other. The bonding bodies 28 that have been attached to graft 24 are received and held within receptacles or open cells at the stent end (preferably, at least the distal end, i.e., the leading end, of the stent graft) formed, for example, by two adjacent bridging elements 26 (e.g., as illustrated along a line as if the stent were cut along dashed line 18 in either of FIGS. 1 or 2.

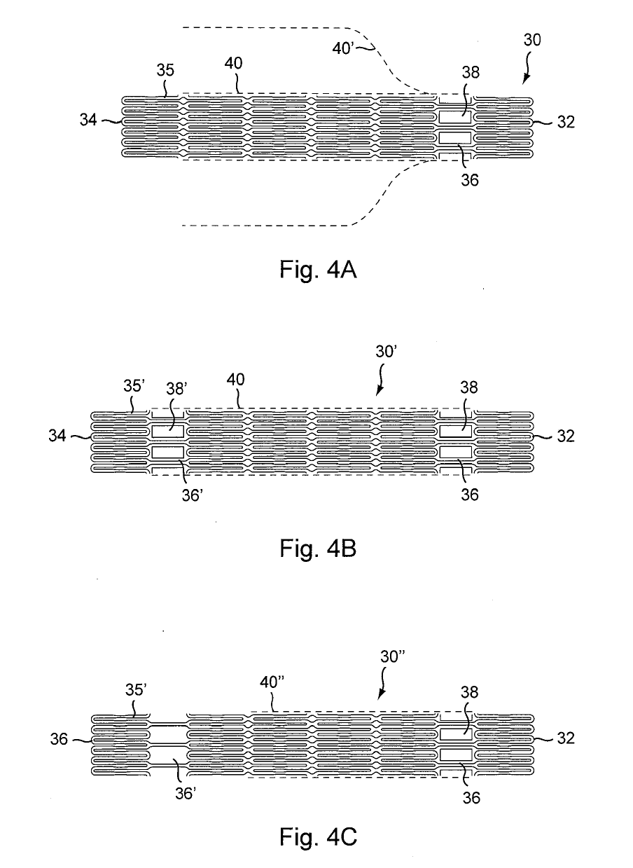

FIGS. 4A-4C are side views of other variations of self-expanding or balloon expandable stent grafts in which their stent lattice structures 35 are pictured in unrolled and compressed configurations with the graft portions shown in phantom. Stent 30 has a distal end 32 and a proximal end 34 with receptacles 36 positioned towards the distal end 32 of the stent for receiving bonding bodies 38. The graft is shown in various relations to the stent. Graft 40 is the graft as it might be placed for a balloon expandable stent, closer fitting with very little loose material. Graft 40′ depicts the graft material as it might be configured for a self-expandable stent.

The extra bulk of the material of graft 40′ may be folded with longitudinal pleats (not shown), similar to the manner in which a percutaneous angioplasty balloon is folded around a stent delivery sheath, as referenced above. Even in a self-expanding version, without longitudinal pleats, the distal end or graft 40′ may be sized to the compressed stent diameter, thereby requiring balloon dilation to effect final deployment after delivery system release.

Stent graft 30′ of FIG. 4B is of similar construction to stent graft 30 of FIG. 4A with stent 35′ having distal end 32, proximal end 34 and distal receptacles 36, with the addition of proximally positioned receptacles 36′ for receiving bonding body blocks 38′. As such, graft 40′ is coupled to both ends of stent 35′ by the function of the two sets of receptacles and blocks.

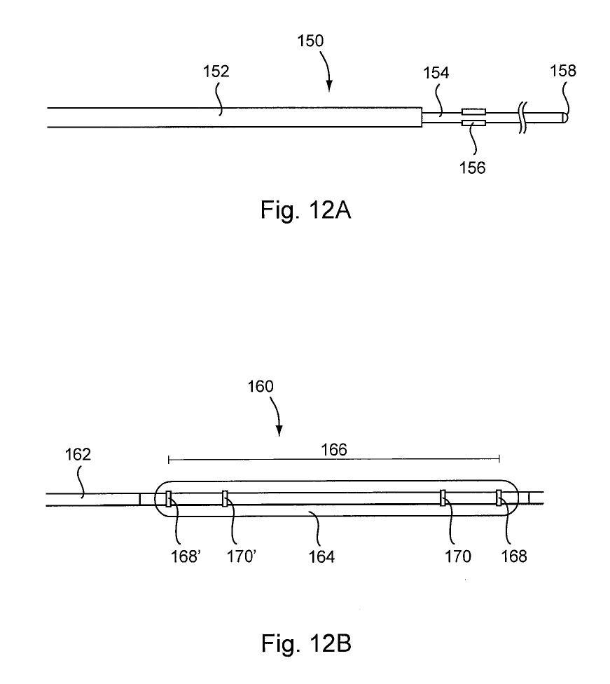

In stent graft 30″ of FIG. 4C, the proximal set of receptacles 36′ in stent 35′ are not used to attached the graft. Rather, graft 40″ extends over less of the length of stent 35′ and is attached thereto with just the block-receptacle set pair at distal end 32. The unused receptacle regions 36′ at proximal end 36 of the stent are reserved to function as an anti-jump feature in which in conjunction with a delivery pusher 150 such as shown in FIG. 12A in receipt of blocks 156 until released as discussed further below.

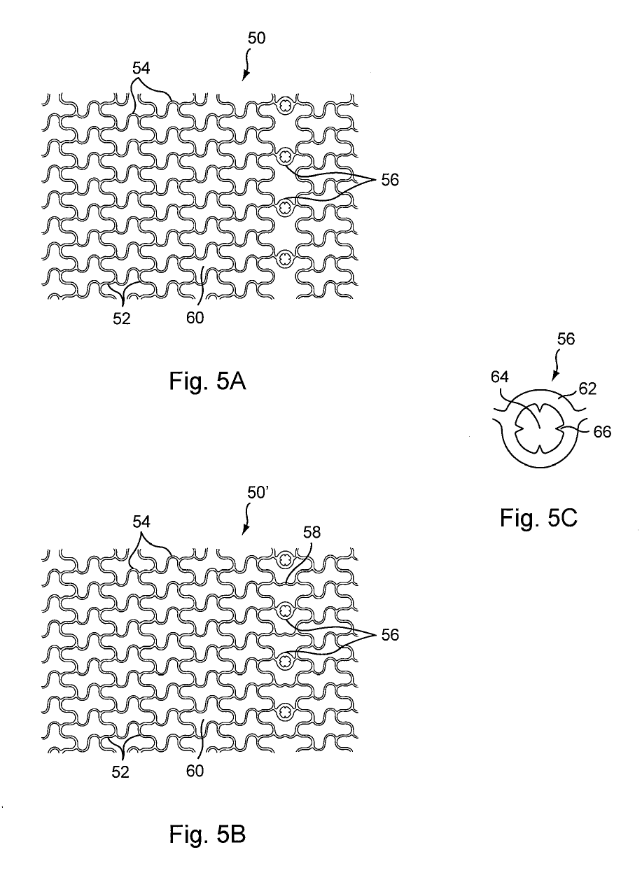

FIGS. 5A and 5B illustrate other stent lattice structures suitable for use with the stent graft inter-retention features of the present invention. Both cell patterns 50 and 50′ represent closed cell designs formed by parallel rows of serpentine struts 52 interconnected by S-shaped bridging members 54. Towards at least one end of the stent structure, a row of spaced apart eyelets 56 interconnects the struts. Pattern 50 incorporates additional bridges 58 between the eyelets. These may offer additional support to the graft when the implant is expanded. However, pattern 50 (without bridges 58) may be useful in achieving smaller compressed delivery profiles in avoiding potential interference or contact with eyelets 56.

As illustrated in the enlarged view of FIG. 5C, an eyelet 56 generally comprises an outer rim 62 defining an open space 64 in the middle to define the receptacle region. As mentioned previously, rim 62 may have any shape best suited for retaining the attachment body element to be received therein. Particularly where interference-type bodies are to be used, the inner profile of rim 62 may have one or more protrusions 66 to enhance the engagement with the press-fit body and/or graft material.

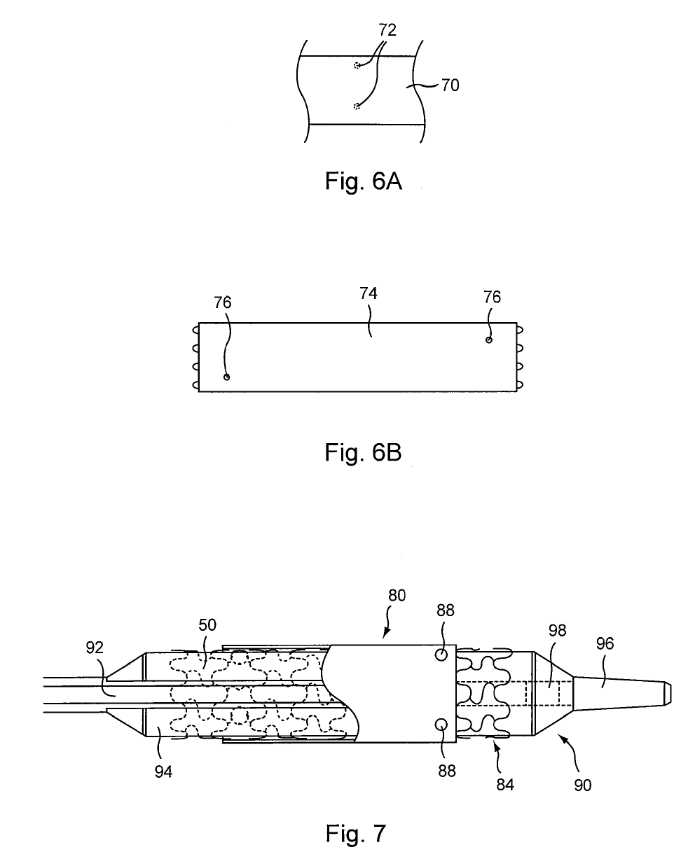

FIGS. 6A and 6B illustrate stent grafts of the present invention utilizing stents having eyelet-type receptacles therein. In FIG. 6A, the eyelets retain polymer block bonding bodies 72 (shown in phantom) which have been heat-bonded to the inside of the graft material of stent graft 70. In FIG. 6B, pins or rivets type interference bodies 76 depress and capture the graft material into eyelets at distal and proximal ends of stent graft 74. Although not necessary, the interference bodies may be made of a radiopaque material to provide a marking function.

FIG. 7 provides a view of a stent graft 80 incorporating a graft 82 attached to a stent 84 of the pattern shown in FIG. 5B by attachment bodies 88. The stent graft is illustrated in an expanded condition over a balloon delivery catheter system 90. The delivery system includes balloon catheter 92 having balloon 94 and terminating distally in atraumatic distal tip 96. The distal portion of catheter 92 may be further provided with a radiopaque marker 98 to identify the distal end position of balloon and/or implant 94 during delivery and deployment. Radiopaque rivets 88 are also used for the inter-retention of stent graft 80, as discussed above with respect to FIG. 6B.

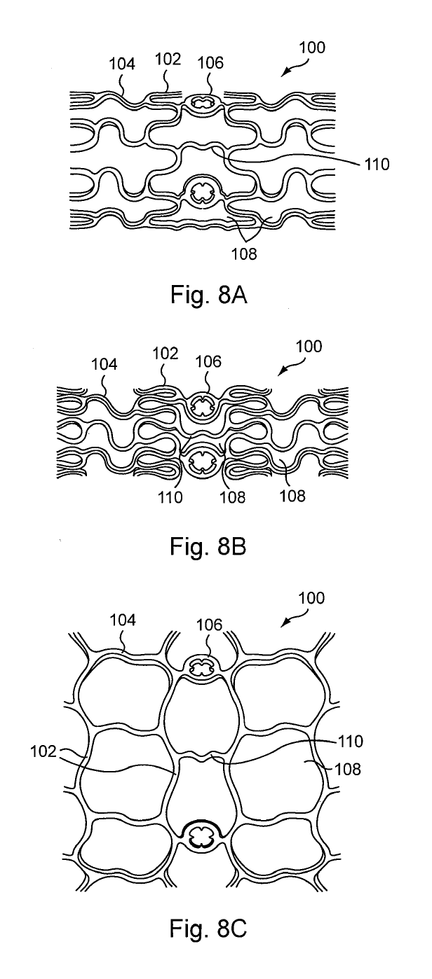

In addition to the graft-to-stent retention features, the present invention includes a novel stent design. This is best illustrated in FIGS. 8A-8C. FIG. 8A illustrates the stent pattern 100 in its original configuration upon being cut from tubing. The pattern includes a series of rows of serpentine struts 102 interconnect to each other near adjacent apices by bridging elements 104 to form a plurality of cells 108. The bridging connection elements 104 bow in the same direction throughout the pattern. The pattern may also includes one or more rows (only one is illustrated) of retention eyelets 106 for the purposes discussed above. Further optional bridging elements 110 may alternate between the eyelets.

The stent, when operatively loaded onto a delivery catheter, is radially compressed or crimped, as illustrated in FIG. 8B. When in the compressed condition, the axial portions of the struts 102 closely pack with one another. Yet, the bridge segments are substantially free to flex axially to easy tracking and delivery through tortuous anatomy.

For implantation, the stent pattern 100 is expanded to a configuration substantially as illustrated in FIG. 8C. In this configuration, the highly asymmetrical cells shown in FIG. 8A and (even more dramatically) in FIG. 8B take on a symmetrical rhomboidal shape (with the exception of eyelet rows) with individual strut segments that are close to or identical to each other in length and shape. The straightened S-shaped members produce a newly-consistent pattern that offers uniform coverage and dynamic support to the graft material that may surround the stent. Adjacent rows of the rhomboidal cells attach to one another, at an offset. A spiral-type pattern (extrapolated from the sections shown in Fig 8C) is thereby produced. The offset or spiral offers additional aide in the support role of the stent (for the graft or bare in supporting a lesion) by avoiding rows/series of cells prone to biased performance along discrete lines.

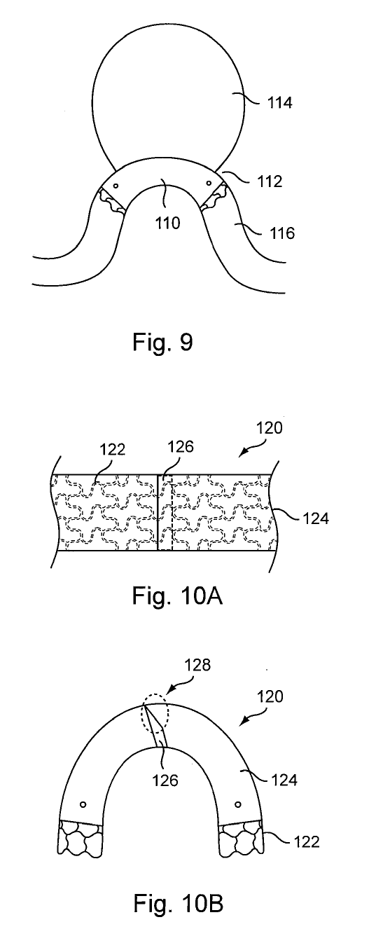

FIG. 9 illustrates a stent graft according to any of a variety of aspects of the invention as it bridges across the neck 112 of aneurysm 114 bulging from a vessel wall 116. The present invention also provides novel graft arrangements that are particularly advantageous to accommodate tightly curved or tortuous implant sites such as shown in FIG. 9

More particularly, FIG. 10A illustrates a section a stent graft 120 in which a portion of the graft material 124 has been folded back upon itself over the stent 122 to form a pleat 126. The overlapping section of the pleat will typically range from about 1 mm to about 5 mm, depending on implant size. As illustrated in FIG. 10B, pleat 126 is able to unfold or un-furrow along the outer extent 128 of the stent graft 120 when placed in a curved position. Such action alleviates strain otherwise placed on the graft material due to the difference in length of the inside and the outside of the curve to which the graft is expected to conform.

One or more such folds or pleats may be provided in the graft material to accommodate the various locations which a stent graft may be subject to higher strains while still maintaining the position of the graft ends relative to the stent structure. One advantageous configuration places a single pleat in the center of the implant. Another (not shown) includes one closer to each end, but inboard of the graft attachment bodies.

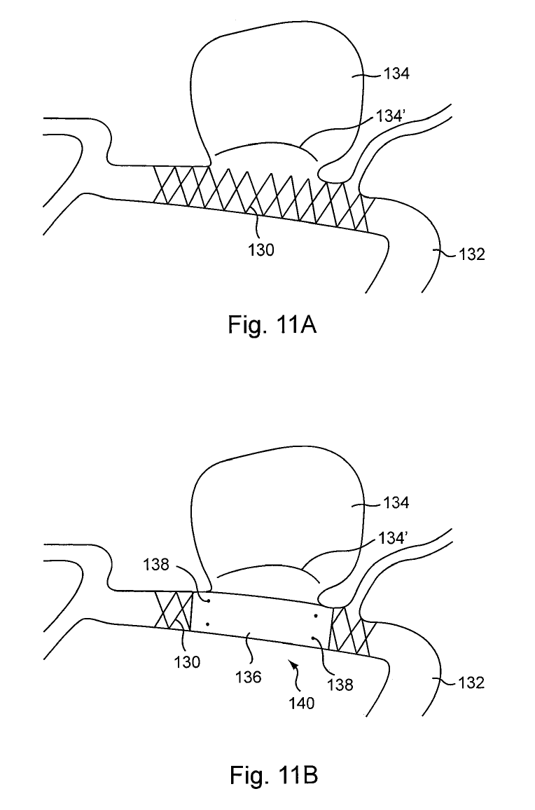

The various stents and stent grafts of the present invention are useful to treat aneurysms and stenotic vessels and are deliverable in the numerous conventional ways known to those skilled in the art of stent delivery. For example, FIG. 11A illustrates the placement of a bare expanded stent 130 within a vessel 132 across the neck 134′ of an aneurysm 134. Such a device might be used to subsequently “jail” coils to achieve high packing density with the aneurysm.

FIG. 11B depicts a stent graft 140 comprising a stent 130 and graft 136 placed across aneurysm 134 occluding the aneurysm neck 134′ in vessel 132. Attachment bodies 138 (in this case, radiopaque interference bodies) hold graft 136 onto stent 130 and also serve to locate the stent graft 140 position relative to the aneurysm neck 134′ as visualized by medical imaging during implant placement.

The stent graft depicted in FIG. 11B is an example of a “hemi” stent graft where some of the stent 130 extends beyond the graft material 136. The extension may be at one end only or at both ends to assist in anchoring without significant obstruction of adjacent vessels as shown.

FIG. 12A depicts a self expanding stent delivery system 150 including a catheter or sheath 152 and a pusher/core member 154. The pusher may include a simple shoulder (not shown) or interface members 156 adapted to interfit with receptacles in the stent (per the discussion above regarding the device in Fig. 4C). Pusher 154 may terminate at the blocker interface or extend under some or the whole length of the implant (not shown). In either case, it may include an atraumatic tip at its end 158. Moreover, pusher 154 may include a full or partial guidewire lumen for “over-the-wire” or “rapid exchange use” as understood by those with skill in the art, respectively.

FIG. 12B depicts a balloon catheter delivery system 160 for a balloon expandable implant (not shown). It includes a catheter body 162 carrying a balloon 164 upon which an implant is mounted over a region 166 advantageously indicated by outboard radiopaque markers 168 and 168′ to indicate the implant ends. Graft ends may also be indicated by inboard radiopaque markers 170. The balloon catheter system will generally be configured for over-the-wire or rapid-exchange use. Alternatively, the balloon catheter system can be a “fixed tip” balloon system that terminates in an atraumatic tip (not shown).

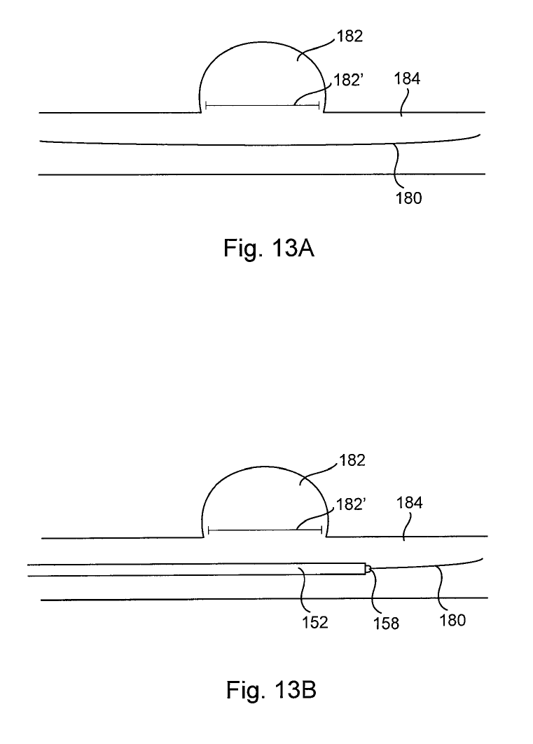

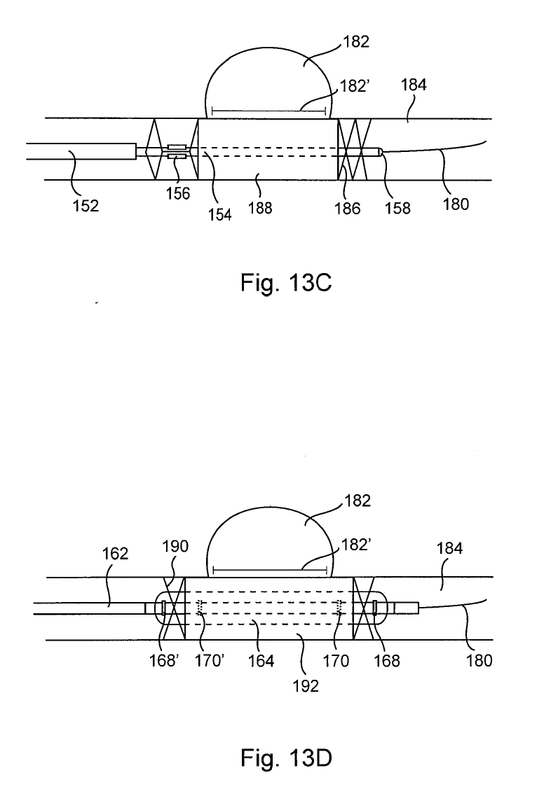

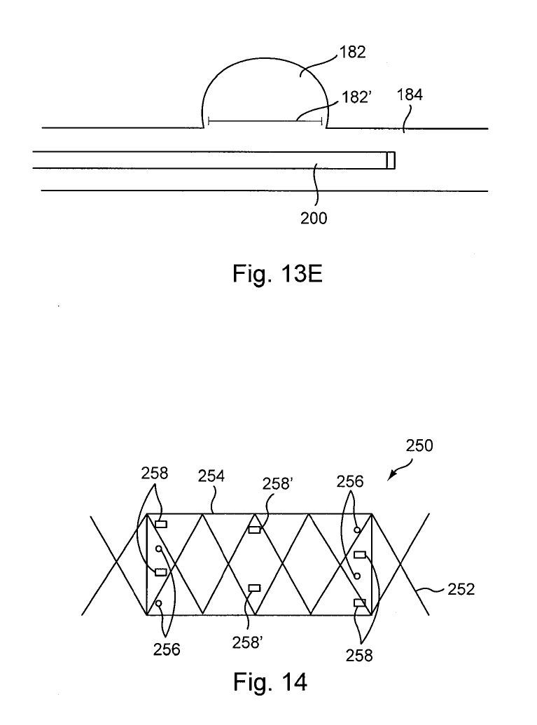

FIGS. 13A-13E illustrate acts performed during delivery and deployment of a stent graft when using the delivery systems illustrated in FIGS. 12A and 12B. FIG. 14A depicts a guidewire 180 advanced to a target site in a vessel lumen 184 for facilitating delivery system navigation to the aneurysm 182 and place a device across the aneurysm neck 182′. FIG. 14B depicts the self-expandable stent graft delivery system 150 of FIG. 12A advanced directly over guidewire to aneurysm 182. The delivery sheath 152 holds a self-expanding stent graft within its lumen.

In FIG. 13C, sheath 152 is retracted proximally while pusher 154 remains in position to deploy the stent graft at aneurysm neck 182′. Blocker interface elements 156 retain the position of the stent graft relative to aneurysm 182 and provide controlled release of the stent graft during sheath removal. Until they are uncovered, the implant can also be retrieved back into the delivery sheath. Upon deployment, the self-expanding stent 186 expands in a controlled fashion setting graft covering 188 across and occluding aneurysm neck 182′.

FIG. 13D depicts the balloon-expanding stent graft delivery system 160 of FIG. 13B at the deployment step parallel to that depicted in FIG. 14C for self-expanding delivery system 150. Here, balloon expandable stent 190 covered by graft 192 has been expanded and deployed across aneurysm neck 182′ by balloon 164 (which is shown in a partially deflated condition to allow retraction from the lumen of the implant). Stent position markers 168, 168′ and graft position markers 170, 170′ are shown within balloon 164, but are not necessary.

As mentioned previously, the balloon delivery system may be configured for over the wire or rapid exchange use. As such, the balloon catheter may simply track over the wire past any guide catheter employed. However, using a “telescoping” catheter approach, a guide catheter or large-lumen microcatheter 200 can first be advanced to or past the aneurysm treatment site as illustrated in FIG. 13E. Then, the balloon catheter can be passed through the same taking advantage of what is commonly a PTFE lined lumen. Such an approach may ease device navigation, as well as minimize vessel trauma. If the guidewire is withdrawn, the presence of such a working lumen also facilitates the uses of a fixed-tip balloon and any further crossing profile reduction advantages it may offer (i.e., in addition to those of the graft attachment systems described herein).

As for other implant variations, FIG. 14 illustrates a stent graft 250 in which graft 254 partially covers and is attached to stent 252 at distal, proximal and medial locations. The graft material is attached at distal and proximal ends with interference bodies 256. The graft is also secured to the stent at distal and proximal ends with bonding bodies 258. In addition, to check the possibility of a portion of the graft “billowing” out of contact with the stent struts (e.g., when across the neck of an aneurysm when not opposed by tissue) bonding bodies 258′ are secured at one or more points in the medial portion of the stent graft 250.

Alternatively, or additionally, radiopaque interference bodies may be employed is along the graft. However, it may be preferred that any medial/intermediate graft attachment is accomplished without adding radiopacity. Avoiding the same may alleviate confusion regarding graft end location (a possibility, especially if the ends of the stent also include radiopaque features). In some examples (e.g., when the entire stent scaffold is covered by graft), none of the attachment bodies are radiopaque—thereby allowing the radiopacity inherent to stent to exclusively indicate graft coverage. Other implant feature sets/configurations are possible as well.

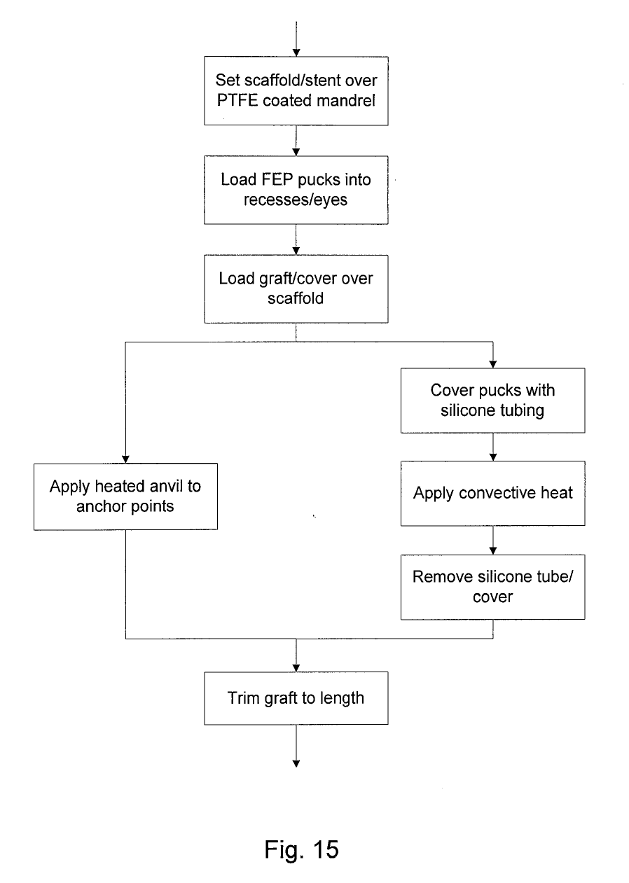

Methods of fabrication of the subject implants are also provided. The flowchart of FIG. 15 illustrates the core steps of one such process for interconnecting the stent and graft with polymer bonding bodies. After the scaffold material is cut and formed into a tubular stent, and the graft material separately has been prepared, the stent is set over a PTFE-coated mandrel. FEP pucks/blocks are then placed within the designated eyelets or recesses of the stent scaffolding over which the graft material is placed. Various heat treatments may be employed to melt the pucks/blocks to affix the stent and graft material together. One method (shown on the left side of the flowchart) employs a heated anvil, such as a temperature-controlled soldering iron, applied to the graft material over each of the polymer puck locations. Another method (shown on the right side of the flowchart) involves the use of convective heat. Prior to apply the heat, silicone tubing is positioned about the construct in order to compress the stent, graft and polymer pucks together. Hot air having a temperature of about 550° F. is then applied (either from inside or outside the stent or both, depending on the exact configuration of the heat bonding device being used) to melt the polymer pucks sufficiently to heat-bond the pucks to the stent and graft. Upon cooling, compressive silicone sheath is removed and the stent and graft material are bonded together.

The graft material may then be trimmed to the desired length. In an alternative approach graft is first trimmed to length. The same may be true of press-fitting approaches to the graft attachment. Trimming may be performed before or after graft affixation. Such trimming may be performed manually, with any type of cutter (e.g., a razor blade) mounted to circumnavigate the implant or by a cutter held stationary while rotating the graft against the blade as one would employ a lathe.

Other acts known to those skilled in the art for fabricating and treating the stent, graft, and stent graft may be employed as necessary and desired. For example, one or more folds or pleats may be made within the graft material prior to heat treating.

Also included in the invention are kits including the various constituent parts of the systems and those that would inter-fit with them to provide the functionality described. These may be provided in packaged combination, gathered by an end-user at a hospital site, etc.

The invention includes methods that may be performed using the subject devices or by other means. The methods may all comprise the act of providing a suitable device. Such provision may be performed by the end user. In other words, the “providing” (e.g. placing the implant at the neck of a cerebral aneurysm in a patient) merely requires that the end user obtain, access, approach, position, set-up, activate, power-up or otherwise act to provide the requisite device in the subject method. Methods recited herein may be carried out in any order of the recited events which is logically possible, as well as in the recited order of events.

Also, it is contemplated that any optional feature of the inventive variations described may be set forth and claimed independently, or in combination with any one or more of the features described herein. Reference to a singular item, includes the possibility that there is a plurality of the same items present. More specifically, as used herein and in the appended claims, the singular forms “a,” “an,” “said,” and “the” include plural referents unless specifically stated otherwise. In other words, use of the articles allow for “at least one” of the subject item in the description above as well as the claims below. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

Without the use of such exclusive terminology, the term “comprising” in the claims shall allow for the inclusion of any additional element--irrespective of whether a given number of elements are enumerated in the claim, or the addition of a feature could be regarded as transforming the nature of an element set forth in the claims. Except as specifically defined herein, all technical and scientific terms used herein are to be given as broad a commonly understood meaning as possible while maintaining claim validity.

The breadth of the present invention is not to be limited to the examples provided and/or the subject specification, but rather only by the intended scope of the following claims.

1. An tubular prosthesis having a longitudinal axis, the prosthesis including a stent comprising:
a plurality of cells wherein the cells have an asymmetrical configuration about the longitudinal axis when the stent is in a first state and a rhomboid configuration when the stent is in an second, expanded state.

2. The prosthesis of claim 1, further comprising a graft material around an outer circumference of at least a portion of the stent; and
at least one attachment body retained within the stent, wherein the graft is affixed to the stent by the attachment body.

3. The prosthesis of claim 2, wherein the attachment body is a polymer body retained within a cell of the stent, wherein the graft is heat-bonded to the polymer body.

4. The prosthesis of claim 2, wherein the attachment body is a metal body retained within an eyelet, wherein the graft is retained by an interference fit between the eyelet and the metal body.

5. A stent graft comprising:
a stent comprising a tubular support structure;
a graft disposed around at least a portion of a length of the stent; and
a plurality of bodies received at least substantially completely within the support structure, wherein the graft is secured to the stent by the bodies alone.

6. The stent graft of claim 5, wherein the support structure provides receptacles for the bodies only when in an unexpanded state.

7. The stent graft of claim 6, wherein the bodies are polymer bodies that are heat bonded to the graft, thereby holding the graft to the stent.

8. The stent graft of claim 5, wherein at least one body forms an interference fit between the graft and the support structure, capturing and holding the graft to the stent.

9. The stent graft of claim 8, wherein the stent comprises eyelets which are present in the stent in both an expanded and unexpanded state, the eyelets receiving bodies adapted for holding the graft to the stent.

10. The stent graft of claim 5, wherein the stent is balloon-expandable.

11. The stent graft of claim 5, wherein the stent is self-expandable.

12. The stent graft of claim 5, wherein the graft is secured at a distal end.

13. The stent graft of claim 12, wherein the graft is secured at a proximal end.

14. The stent graft of claim 5, wherein the graft includes a pleat.

15. A method of retaining a graft to a stent, the method comprising:
setting a stent over a mandrel,
loading receptacles in the stent with polymer bodies,
overlaying a graft, and
heat bonding the polymer bodies to a graft material.

16. The method of claim 15, wherein the bonding is performed with a heated anvil applied on an outer surface of the graft.

17. The method of claim 15, wherein the method further comprises:
covering at least the region of the stent receiving polymer bodies with silicon tubing,
applying convective heat, and
removing the silicon covering.

18. A method of making a stent graft comprising:
positioning a graft around at least a portion of a support structure, the support structure defining receptacles for receiving attachment bodies; and
securing the graft to the support structure with attachment attachment bodies, without the bodies protruding beyond the receptacles.

19. The method of claim 18, the securing of the graft comprising connecting at least one of the bodies in the receptacles to the graft by an interference fit.

20. The method of claim 18, the securing of the graft comprising connecting at least one of the bodies in the receptacles to the graft by heat bonding.
